(19) United States
(12) Patent Application Publication
(10) Pub. No.: US 2017/0120729 A1
(43) Pub. Date: May 4, 2017

(54) STRAW AIR FRESHENER WITH CAP

(71) Applicant: Energizer Brands II, LLC, St. Louis, MO (US)

(72) Inventor: Kathy Rasmussen, Draper, UT (US)

(21) Appl. No.: 15/337,708

(22) Filed: Oct. 28, 2016

Related U.S. Application Data
(60) Provisional application No. 62/248,042, filed on Oct. 29, 2015.

(51) Int. Cl. B60H 3/00; A61L 9/01

(52) U.S. Cl. CPC B60H 3/0035; A61L 9/01

(57) ABSTRACT

An air freshener comprises a scented material impregnating a porous wall of an elongated hollow straw. A cap is coupled to an end of the straw. A bore extends through the cap and receives a hanger extending therethrough to hang the straw. The cap has a plug extending therefrom and inserted into an end of the straw. The plug is tubular with an elongated hollow continuous with and adjoining a hollow of the straw so that the straw and the plug together define an elongated tube open at both ends.

### CROSS-REFERENCE TO RELATED APPLICATIONS

This patent application claims priority from U.S. Provisional Application Ser. No. 62/248,042, filed Oct. 29, 2015, which is incorporated herein by reference in its entirety.

### BACKGROUND

Field of the Disclosure

The present disclosure relates generally to a straw type air freshener.

Description of Related Art

Various different types of air fresheners have been developed, particularly for use in vehicles. Vent stick type air fresheners include an aromatic body carried by the air vent, and utilizing air flow therefrom to disperse scent from the body. A stick-like air freshener has been proposed in which paper is rolled into a solid stick and infused with scent. See AutoSticks fragrance infusers by Enviroscent.

### BRIEF SUMMARY

Example embodiments are directed to air fresheners for vehicles, and other areas. The development of air fresheners is an ongoing endeavor.

Various embodiments are directed to an air freshener with a scented material impregnating a porous wall of an elongated hollow straw. A cap is coupled to one end of the straw. A bore extends through the cap and receives a hanger extending therethrough to hang the straw. The cap has a plug extending therefrom and inserted into the one end of the straw. The plug is tubular with an elongated hollow continuous with and adjoining a hollow of the straw so that the straw and the plug together define an elongated tube open at both ends.

Certain embodiments are directed to an air freshener device comprising a tubular body comprising one or more walls, wherein the one or more walls define a hollow interior passage extending between a first open end of the tubular body and a second open end of the tubular body. In various embodiments, the one or more walls are impregnated with a scented material. The air freshener device may additionally comprise a cap having a plug portion extending therefrom, wherein the plug portion is interference-fit within the hollow interior passage to secure the cap relative to the tubular body.

In certain embodiments, the cap additionally defines a bore extending therethrough; and the air freshener device additionally comprises a hanger extending through the bore, wherein the hanger is configured to suspend the air freshener from an object. Moreover, in certain embodiments, the plug defines one or more protrusions extending therefrom. The protrusions may be configured to engage the interior surface of the one or more walls to impede movement of the cap relative to the tubular body.

In various embodiments, the tubular body is at least substantially cylindrical. Moreover, the cap may be cylindrical and have an exterior diameter at least substantially the same as the exterior diameter of the tubular body. Moreover, in certain embodiments, the hollow interior passage is at least substantially cylindrical and the plug portion is at least substantially cylindrical, and wherein an exterior diameter of the plug portion is at least substantially the same as an interior diameter of the hollow interior passage. In certain embodiments, the cap defines a cap axial opening extending between a first cap open end and a second cap open end, and wherein the cap axial opening is at least substantially concentric with the hollow interior passage.

Moreover, in certain embodiments, the tubular body may have indicia disposed on an outer surface thereof In various embodiments, the air freshener comprises a decorative outer layer disposed around the tubular body. In certain embodiments, the tubular body portion comprises paper. The tubular body portion may comprise a plurality of paper layers. Moreover, in certain embodiments, the cap comprises a metal or plastic material. In various embodiments, the tubular body portion comprises a material different from the cap.

### BRIEF DESCRIPTION OF THE SEVERAL VIEWS OF THE DRAWINGS

Reference will now be made to the accompanying drawings, which are not necessarily drawn to scale, and wherein:

FIG. 1 shows a perspective view of an air freshener according to various embodiments;

FIG. 2 shows a close-up exploded view of a portion of an air freshener according to various embodiments;

FIG. 3 shows a perspective exploded view of an air freshener according to various embodiments;

FIG. 4 shows a side view of a suspended air freshener according to various embodiments;

FIG. 5 shows a top view of a cap of an air freshener according to various embodiments; and

FIG. 6 shows a perspective view of an air freshener straw according to various embodiments.

### DETAILED DESCRIPTION

Aspects of the present disclosure will now be described more fully hereinafter with reference to the accompanying drawings, in which some, but not all embodiments are shown. Indeed, various embodiments may be embodied in many different forms and should not be construed as limited to the embodiments set forth herein. Rather, these embodiments are provided so that this disclosure will satisfy applicable legal requirements. Like numbers refer to like elements throughout.

### Definitions

The terms “top” and “bottom” and “downwardly” and “upwardly” and the like are used herein relative to the air freshener device or housing thereof being oriented upright or vertical; while it is understood that the device or housing can be oriented horizontally or at an incline during use.

The term “scent material” and “fragrant material” are used interchangeably herein to refer broadly to a material (e.g., liquid, powder, gel, solid, gas, and/or the like) providing a desired fragrance, scent, scent neutralizing agent, and/or the like. In various embodiments, the scent material may be a volatile material configured to evaporate and/or sublimate to disperse within a gaseous (e.g., air) environment.

The terms “interference-fit” and “friction-fit” and “press-fit” are terms of art used interchangeably herein to refer to deliberately causing, increasing and/or using friction to deliberately resist movement. An interference-fit or friction-fit or press-fit is different than, and great than, the existence of friction. While friction may exist between any two surfaces, is often desirable to do all one can to reduce this friction. An interference-fit or friction-fit or press-fit can be distinguished from naturally occurring friction by being actually deliberately caused and increased. An interference-fit or press-fit can be created by dimensioning engaging parts so that their surfaces tightly bear against one another. In various embodiments, a friction fit may be provided between a first object having a male connector and a second object having a corresponding female connector. In various embodiments, the exterior dimensions of the male connector may be identical to and/or slightly larger than the interior dimensions of the corresponding female connector, such that at least a portion of the exterior sides of the male connector tightly engage the corresponding interior sides of the female connector to form a tight, interference fit. However, it should be understood that in various embodiments, the exterior dimensions of the male connector may be slightly smaller than the interior dimensions of the corresponding female connector while forming a tight interference fit.

### Description

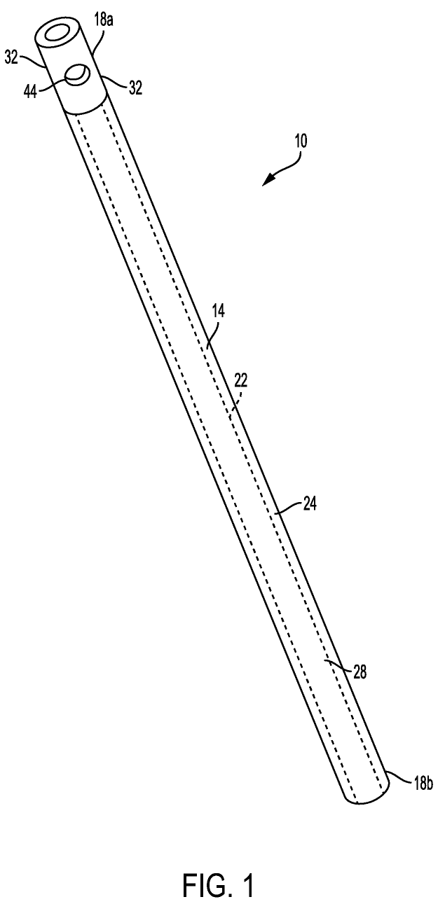

As illustrated in the Figures, an air freshener, indicated generally at 10, in an example implementation in accordance with various embodiments is shown. As shown in FIG. 1, which illustrates an assembled air freshener 10 according to one embodiment, the air freshener 10 may have a tubular configuration, with interior and exterior surface areas, to increase the dispersal of scent material. In various embodiments, the air freshener may have a cylindrical configuration, a rectangular tubular configuration, a triangular tubular configuration, and/or the like. The air freshener 10 may be configured to be suspended from a hanger, for example, in a vehicle, a room, a closet, and/or other desired area. In addition, in the embodiment shown in FIG. 1, the air freshener 10 may have a cap to resist leaching of scent material embedded or otherwise contained within a body portion of the air freshener 10.

The air freshener 10 may comprise an elongated tube or tubular body 14 comprising one or more walls (e.g., cylindrical wall 28) and having opposite ends 18a and 18b and defining a central axis extending parallel to the elongated tube between opposite ends 18a, 18b. In various embodiments, the elongated tube 14 may be linear (as shown in FIG. 1), however it should be understood that in various embodiments, the elongated tube 14 may be curved, kinked, angled, and/or the like. As shown in FIG. 1, one or more of the opposite ends 18a, 118b may have associated openings. For example, opposite ends 18a, 18b may define a first open end extending into the tubular body 14 and a second open end extending into the tubular body 14. In various embodiments, the first open end and the second open end may extend into the tubular body 14 in a direction at least substantially parallel with the central axis of the air freshener 10. In various embodiments, one or more of the openings may be concentric with one another and/or the tubular body 14. In various embodiments, the tubular body 14 may define an elongated hollow interior passage 22 extending the length of the tube and between the openings (e.g., along the central axis). In various embodiments, the elongated hollow interior passage 22 may be concentric with the tubular body 14 and may be defined between the one or more walls. In various embodiments, the air freshener 10 may have a length (measured along the central axis) much greater than a diameter (measured perpendicular to the elongated axis). As just one non-limiting example, the length can be greater than ten times the diameter. In various embodiments, the elongated hollow interior passage 22 increases the surface area of the air freshener and the openings in the ends enable scent to emanate from the elongated hollow interior passage 22. As will be discussed herein, scent material may be embedded in, impregnated in, or otherwise contained within the tubular body 14 (e.g., within the walls of the tubular body 14). The scent material may be a volatile material configured to evaporate, sublimate, and/or the like from the surface of the elongated body 14 into the air such that the scent material provides a desired scent in an environment surrounding the air freshener 10. Accordingly, an increased surface area (e.g., by presenting a surface area within an elongated hollow interior passage 22 to a surrounding environment) may permit a greater simultaneous mass transfer of scent material to the surrounding environment (e.g., via evaporation and/or sublimation).

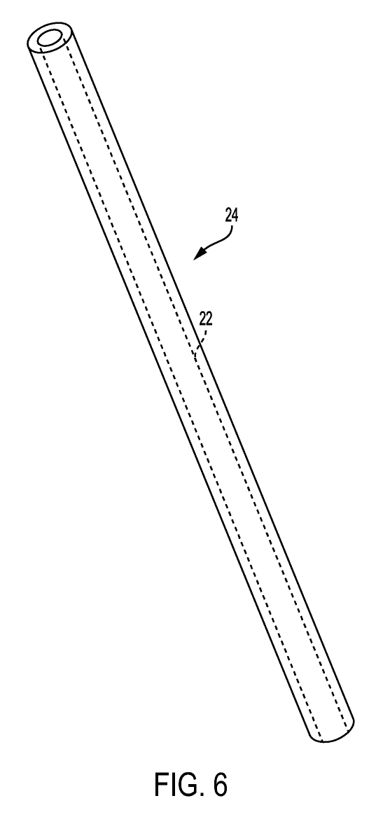

In various embodiments, the air freshener 10 may comprise an elongated hollow tubular straw 24 as shown in FIG. 6. Like the tube or tubular body 14, the straw 24 may be elongated with open opposite ends and a hollow interior extending therein (e.g., between open opposite ends). The straw 24 may define a cylindrical wall 28. The wall may be porous and/or fibrous, and/or may be a porous or fibrous wall. In various embodiments, the wall 28 may comprise a woven material, a non-woven material, a fibrous material, and/or the like. For example, the wall 28 may comprise a paper (e.g., a fibrous paper), a plastic, a fabric, and/or the like. In one aspect, the wall 28 may comprise one or more layers (e.g., a plurality of wound layers). In various embodiments, the wall 28 may comprise porous paper that is straight wound with a hollow therein. In another aspect, the wall may be a single layer, or may be formed of a single layer of the material having a single exterior surface, a single interior surface, and without any exposed intermediate surfaces. Accordingly, the wall may have a thickness (e.g., measured radially in a direction parallel with a diameter) through the wall that is continuous and uninterrupted by any boundary layers, or changes in material or characteristics. A scented material (and/or neutralizing agent), such as a scented oil, may impregnate the porous wall 28 of the elongated hollow straw 24.

In one aspect, the straw 24 may be formed by wrapping or winding a sheet material (e.g., paper) around a mandrel. In another aspect, the straw 24 may be formed by extrusion, and the straw 24 may be an extruded straw. In another aspect, the straw 24 may be formed of a single sheet of material with opposite ends joined together to form a tube. In one aspect, the scented material may impregnate the porous wall 28 of the straw 24 after the material (e.g., paper) has been formed into a cylindrical configuration (e.g., after a sheet material has been wrapped to form the straw 24). In another aspect, the material may be impregnated with the scented material prior to being formed into the cylindrical configuration.

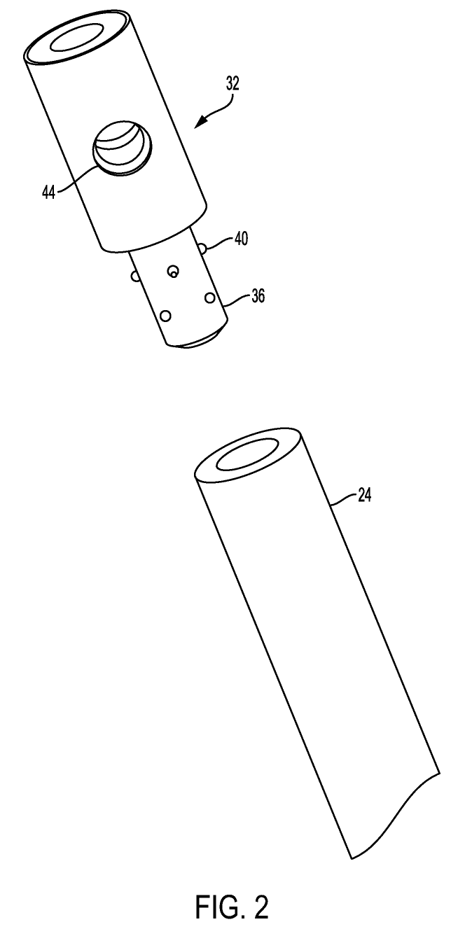

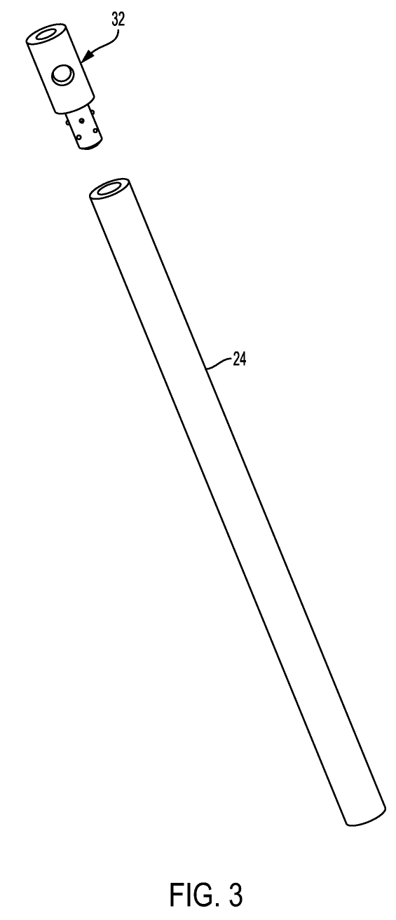

As shown in FIG. 1, the air freshener may have a cap 32 securable relative to one end of the straw 24. In one aspect, the cap 32 may be coupled relative to an interior of the straw 24 (e.g., elongated hollow interior passage 22). The cap 32 can be formed of and comprise a different material than the straw 24. For example, the cap 32 can formed of plastic or metal, however the cap 32 may be formed of any of a variety of materials (e.g., rubber, wood, and/or the like). The material of the cap 32 may resist wicking or leaching of the scent material from the straw 24, through the cap 32 (and to a hanger 48). Thus, objects from which the air freshener 10 is suspended may be protected from the scent material. As shown in FIGS. 2-3 which show exploded views of the air freshener 10, the cap 32 may have a plug portion 36 extending therefrom and inserted into the one end or opening of the straw 24. In one aspect, the plug portion 36 can be press-fit or friction-fit or interference-fit into an opening of one end of the straw 24. In one aspect, the plug portion 36 has one or more protrusions 40 extending radially outwardly therefrom to engage or bear against an interior surface of the straw 24. Accordingly, the plug portion 36 may form a male-connector configured to engage an interior portion of the straw 24, which forms a corresponding female connector for the plug portion 36.

In one aspect, the cap 32 and the straw 24 can have exterior or outer surfaces that are flush with one another. For example, the cap 32 and the straw 24 may have outer diameters that are at least substantially the same. Thus, the assembled cap 24 and straw 32 may be easily inserted and removed from packaging, and may resist snagging or otherwise getting caught on other objects or edges thereof. The plug portion 36 may have a diameter less than a diameter of the straw 24 and the cap 32. Thus, the plug portion 36 can be stepped, to provide for the exterior or exposed surfaces of the cap 32 and the straw 24 to be flush. In various embodiments, the plug portion 36 may have a diameter substantially the same as the diameter of the elongated hollow interior passage 22 to facilitate an interference fit between the plug portion 36 and the straw 24. However, in various embodiments, the plug portion 36 may have a diameter larger than the diameter of the elongated hollow interior passage 22. In yet other embodiments, the plug portion 36 may have a diameter smaller than the diameter of the elongated hollow interior passage 22 while enabling an interference fit therebetween.

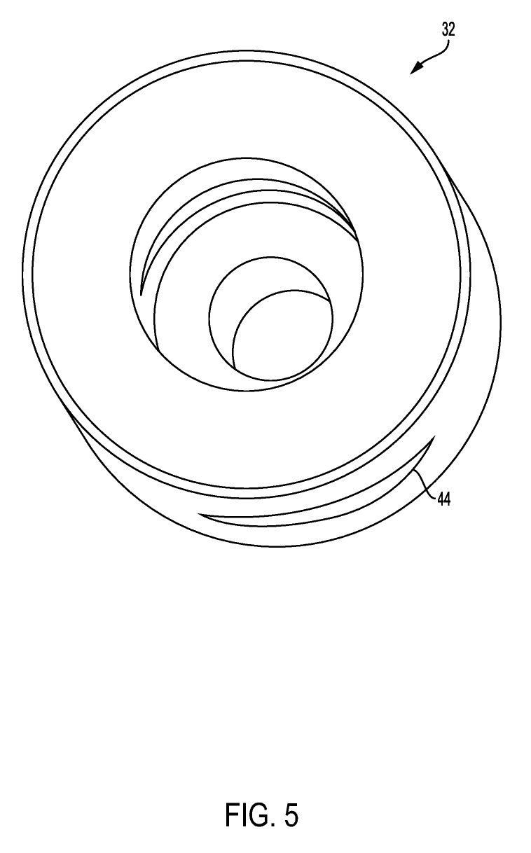

The cap 32 and the plug portion 36 may be tubular with an elongated hollow continuous with and adjoining the elongated hollow interior passage 22 of the straw, so that the straw 24 and the plug portion 36 together define the elongated tube open at both ends (as shown in FIG. 5, which provides a top view of a cap 32 according to various embodiments). Thus, the cap 32 and plug portion 36 can hang the straw 24 therefrom, without closing the opening in the straw 24 or the elongated hollow interior passage 22 therein, and to allow the flow of air and the release of scent material. Accordingly, the cap 32 may define a cap central axis extending along a length thereof (e.g., substantially parallel with a length of the plug portion 36 and the cap 32). The hollow may extend along the length of the cap 32 parallel with the cap central axis.

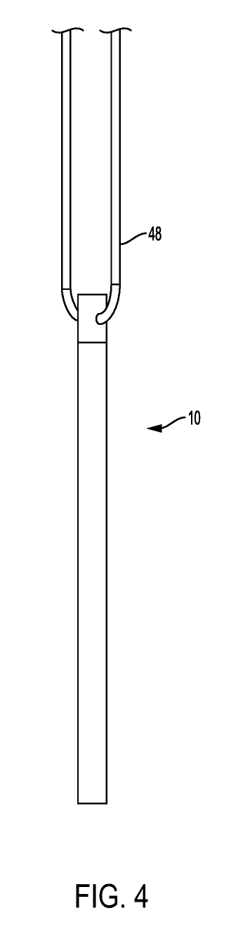

As shown in FIG. 2-3, a bore 44, such as a lateral bore, may extend through the cap 32. In one aspect, the bore 44 may extend laterally through the cap 32, or transverse to a longitudinal axis (e.g., cap central axis and/or central axis) of the air freshener 10 or the straw 24. As shown in FIG. 4, a hanger 48 may extend through the bore 44 of the cap 32. The air freshener 10, or the straw 24 and the cap 32, may be suspended (or may hang) from the hanger 48. In one aspect, the hanger 48 may be a flexible loop, such as string. However, it should be understood that the hanger 48 may be a rigid material (e.g., a rigid wire, a rigid tube, and/or the like). In various embodiments, the plug portion 36 and the bore 44 may be oriented transverse to one another, and an axis of the straw 24.

In addition, at least a portion of the air freshener 10 (e.g., the straw 24), may have indicia disposed on an outer surface thereof In one aspect, indicia may be disposed on a separate sheet or layer, and may be disposed over and/or around the straw 24. In various embodiments, the indicia and separate sheet or layer may be a decorative outer layer. The separate sheet or layer may comprise printed paper that may be fastened to the straw (e.g., via an adhesive), prior to being infused or impregnated with the scent material or oil. In various embodiments, the separate sheet or layer may comprise a porous material, a fibrous material, and/or the like. In various embodiments, the separate sheet or layer may comprise the same material as the straw 24. However, it should be understood that the separate sheet or layer may comprise a different material from the straw 24.

In various embodiments, the air freshener 10 and the straw 24 may be provided in an air tight container or seal to resist premature release of the scent material, or drying of the straw. In use, the air freshener 10 may be removed from the air tight container to enable the scent material to evaporate into a surrounding environment to provide a desired scent therein.

While the forgoing examples are illustrative of the principles of various embodiments in one or more particular applications, it will be apparent to those of ordinary skill in the art that numerous modifications in form, usage and details of implementation can be made without the exercise of inventive faculty, and without departing from the principles and concepts of the disclosure. Accordingly, it is not intended that the disclosure be limited, except as by the claims set forth below.

### Conclusion

Many modifications and other embodiments of the disclosure set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An air freshener device comprising:
a tubular body comprising one or more walls, wherein the one or more walls define a hollow interior passage extending between a first open end of the tubular body and a second open end of the tubular body;
a scented material impregnating the one or more walls; and
a cap having a plug portion extending therefrom, wherein the plug portion is interference-fit within the hollow interior passage to secure the cap relative to the tubular body.

2. The air freshener device of claim 1, wherein the cap additionally defines a bore extending therethrough; and
wherein the air freshener device additionally comprises a hanger extending through the bore, wherein the hanger is configured to suspend the air freshener from an object.

3. The air freshener device of claim 1, wherein the plug defines one or more protrusions extending therefrom, and wherein the one or more protrusions are configured to engage the interior surface of the one or more walls to impede movement of the cap relative to the tubular body.

4. The air freshener device of claim 1, wherein the tubular body is at least substantially cylindrical.

5. The air freshener device of claim 4, wherein the cap is cylindrical, and wherein the cap has an exterior diameter at least substantially the same as an exterior diameter of the tubular body.

6. The air freshener device of claim 1, wherein the hollow interior passage is at least substantially cylindrical and the plug portion is at least substantially cylindrical, and wherein an exterior diameter of the plug portion is at least substantially the same as an interior diameter of the hollow interior passage.

7. The air freshener device of claim 1, wherein the cap defines a cap axial opening extending between a first cap open end and a second cap open end, and wherein the cap axial opening is at least substantially concentric with the hollow interior passage.

8. The air freshener device of claim 1, further comprising indicia disposed on an outer surface of the tubular body.

9. The air freshener device of claim 1, further comprising a decorative outer layer disposed around the tubular body.

10. The air freshener device of claim 1, wherein the tubular body portion comprises paper.

11. The air freshener device of claim 10, wherein the tubular body comprise a plurality of paper layers.

12. The air freshener device of claim 1, wherein the cap comprises a material selected from: plastic or metal.

13. The air freshener device of claim 1, wherein the tubular body portion comprises a material different from the cap.
